(19) United States
(12) Patent Application Publication
(10) Pub. No.: US 2024/0366910 A1
(43) Pub. Date: Nov. 7, 2024

(54) INFORMATION PROCESSING DEVICE

(71) Applicant: NTT DOCOMO, INC., Tokyo (JP)

(72) Inventors: Akinari SAKAI, Chiyoda-ku (JP); Tetsuya YAMAGUCHI, Chiyoda-ku (JP); Takuya IZUMISAWA, Chiyoda-ku (JP); Yusuke NAKAMURA, Chiyoda-ku (JP)

(73) Assignee: NTT DOCOMO, INC., Tokyo (JP)

(21) Appl. No.: 18/688,844

(22) PCT Filed: Aug. 25, 2022
(86) PCT No.: PCT/JP2022/032091

§ 371 Date: Mar. 4, 2024

(30) Foreign Application Priority Data
Oct. 22, 2021 (JP) 2021-173263

(51) Int. Cl. A61M 21/00

(52) U.S. Cl. CPC A61M 21/00; A61M 2021/005; A61M 2205/50

(57) ABSTRACT

A nudge management device includes: a storage unit configured to store information of a nudge which is a mechanism for prompting a user to voluntarily adopt a desirable behavior; an acquisition unit configured to acquire nudge intervention information which is information associated with intervention of a nudge; and an abrasion deriving unit configured to derive nudge abrasion information indicating a degree of decrease in effect of the nudge associated with the nudge intervention information based on the nudge intervention information and to output the derived nudge abrasion information.

### TECHNICAL FIELD

An aspect of the present invention relates to an information processing device.

### BACKGROUND ART

In Patent Literature 1, a system for promoting behavioral modification of a user by using nudges for prompting the user to voluntarily adopt a behavior which is desirable in individuals or societies based on cognitive biases is described.

### CITATION LIST

### Patent Literature

[Patent Literature 1] Japanese Unexamined Patent Publication No. 2020-140596

### SUMMARY OF INVENTION

### Technical Problem

In behavioral economics, the nudges are considered to be abraded. Here, abrasion of a nudge means that a behavioral modification effect based on the nudge decreases because repeated intervention (recommendation for a user) is performed. In the related art, this abrasion of a nudge has not been appropriately considered.

The present invention was made in consideration of the aforementioned circumstances and an objective thereof is to appropriately represent abrasion of a nudge using a numerical value.

### Solution to Problem

An information processing device according to an aspect of the present invention includes: a storage unit configured to store information of a nudge which is a mechanism for prompting a user to voluntarily adopt a desirable behavior; an acquisition unit configured to acquire nudge intervention information which is information associated with intervention of a nudge; and an abrasion deriving unit configured to derive nudge abrasion information indicating a degree of decrease in effect of the nudge associated with the nudge intervention information based on the nudge intervention information and to output the derived nudge abrasion information.

In the information processing device according to the aspect of the present invention, nudge intervention information which is information associated with intervention of a nudge (information associated with a nudge's intervention in a user) is acquired, and nudge abrasion information indicating a degree of decrease in effect of the nudge is derived and output based on the nudge intervention information. In this way, since the nudge abrasion information is estimated (derived) from the information associated with a nudge's intervention in a user (for example, a nudge intervention frequency), it is possible to appropriately (accurately) represent abrasion of the nudge using a numerical value, for example, when the nudge is abraded (a behavioral modification effect of the nudge decreases) due to repeated intervention of the nudge. As described above, with the information processing device according to the aspect of the present invention, it is possible to appropriately represent abrasion of a nudge using a numerical value.

### Advantageous Effects of Invention

According to the aspect of the present invention, it is possible to appropriately represent abrasion of a nudge using a numerical value.

### BRIEF DESCRIPTION OF DRAWINGS

FIG. 1 is a diagram illustrating a process flow of promoting behavioral modification of a user using a nudge.

FIG. 2 is a diagram illustrating abrasion of a nudge.

FIG. 3 is a functional block diagram of a nudge management device according to a first embodiment.

FIG. 4 is a diagram illustrating calculation of abrasion of a nudge.

FIG. 5 is a diagram illustrating calculation of abrasion of a nudge.

FIG. 6 is a diagram illustrating calculation of abrasion of a nudge.

FIG. 7 is a sequence diagram illustrating an abrasion updating process of a nudge.

FIG. 8 is a functional block diagram of a nudge management device according to a second embodiment.

FIG. 9 is a diagram illustrating generation of an abrasion feature quantity.

FIG. 10 is a diagram illustrating estimation of behavioral modification.

FIG. 11 is a diagram illustrating selection of a nudge.

FIG. 12 is a sequence diagram illustrating a nudge recommending process.

FIG. 13 is a diagram illustrating a hardware configuration of the nudge management device according to the embodiment.

### DESCRIPTION OF EMBODIMENTS

Hereinafter, embodiments of the present invention will be described in detail with reference to the accompanying drawings. In description with reference to the drawings, the same or equivalent elements will be referred to by the same reference signs and repeated description thereof will be omitted.

### First Embodiment

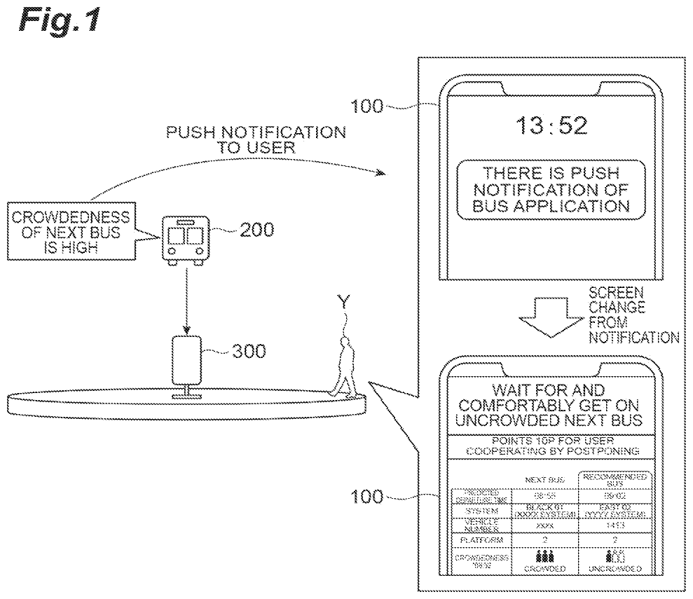

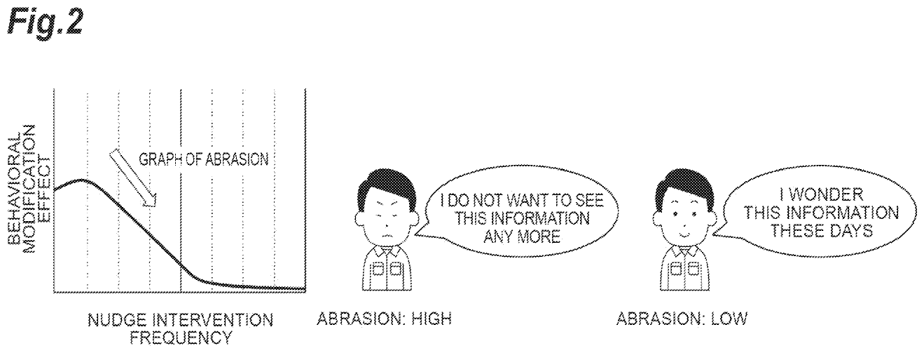

First, a nudge described in a first embodiment and promotion of behavioral modification of a user using the nudge will be described with reference to FIGS. 1 and 2. FIG. 1 is a diagram illustrating a process flow of promoting behavioral modification of a user using a nudge. FIG. 2 is a diagram illustrating abrasion of a nudge.

A nudge is a structure or mechanism based on cognitive biases for prompting a user to voluntarily adopt a behavior which is desirable in individuals or societies. In this embodiment, it is assumed that a nudge is display of a message or the like which is displayed on a communication terminal such as a smartphone. FIG. 1 illustrates a scene in which a user Y moves to a bus stop 300 to get on a bus. As illustrated in FIG. 1, it is assumed that a degree of crowdedness of a bus (a next bus) on which the user Y is scheduled to get is high. In this case, for example, a predetermined application (for example, a bus application for assisting with getting on a bus) causes a communication terminal 100 such as a smartphone of the user Y to output a push notification. The push notification is a notification associated with a nudge for promoting behavioral modification of the user and specifically a notification associated with a nudge for prompting the user to get on a bus (a recommended bus) other than the crowded next bus.

As illustrated in FIG. 1, when the user Y changes the push notification output by the communication terminal 100 to a nudge screen, information indicating that a next bus is crowded and information indicating a scheduled departure time or a degree of crowdedness of a recommended bus are displayed along with a message such as “wait for and comfortably get on an uncrowded next bus” and “points 10p for user cooperating by postponing.” A state in which there has been change to the nudge screen in this way is a “nudge-intervened” state in a user. The user Y checks the nudge screen and selects whether to change an originally scheduled behavior to adopt the behavior of “getting on the recommended bus.”

In order to prompt the user Y to effectively perform behavioral modification (“to get on the recommended bus” in the aforementioned example), it is important to cause a most effectual nudge to intervene in the user Y at an appropriate timing. The appropriate timing at which nudge intervention is performed may be determined, for example, based on information acquired via the application (a “bus application” in the aforementioned example) of the communication terminal 100 or position information of the user Y. In this embodiment, detailed description of the timing at which nudge intervention is performed will be omitted. As an effectual nudge, a nudge with the highest behavioral modification effect in the current scene is considered to be selected, for example, based on information acquired via the application (the “bus application” in the aforementioned example) of the communication terminal 100. For example, in the example in which the scheduled bus is crowded, when there are a nudge for promoting change of a transportation means, a nudge for prompting a user to send the bus off, and a nudge for prompting a user to make a detour, selecting the nudge for prompting a user to send the bus off in which a probability of selection by a user is generally considered to be highest is conceivable.

Here, it is assumed that a behavioral modification effect of a nudge is not fixed but changes in behavioral economics. Specifically, as illustrated in FIG. 2, it is considered that a behavioral modification effect of a nudge having intervened in a user daily (continuously in a long term) decreases gradually. In the following description this decrease of a behavioral modification effect of a nudge based on intervention in a user may be referred to as “abrasion of a nudge.” In a state in which abrasion of a nudge has progressed, a user does not want to see the nudge (information) and it is difficult to promote behavioral modification of the user. On the other hand, in a state in which abrasion of a nudge has not progressed, a user is likely to have an interest in the nudge (information) and it is possible to effectively promote behavioral modification of the user. In this way, since a nudge with a strong behavioral modification effect can be actually selected by deriving nudge abrasion information, it is important to derive nudge abrasion information. Details associated with derivation of nudge abrasion information will be described below in detail.

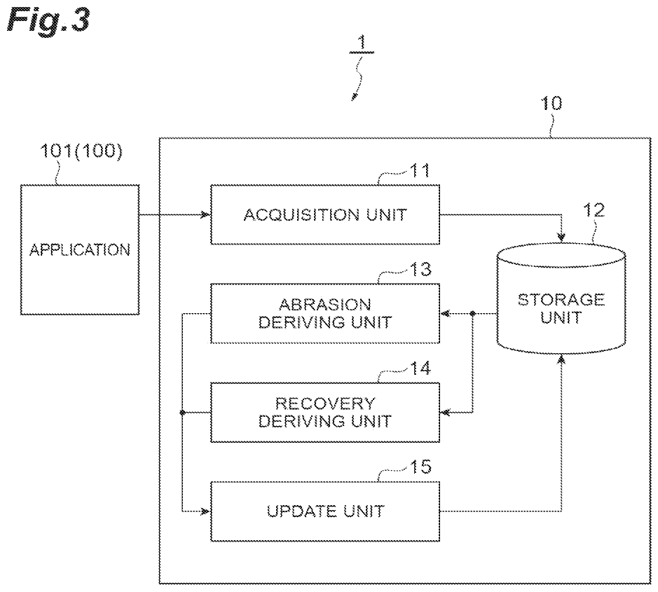

FIG. 3 is a functional block diagram of a nudge management device 10 (an information processing device) included in a nudge recommendation system 1 according to the first embodiment. As illustrated in FIG. 3, the nudge recommendation system 1 includes an application 101 of a communication terminal 100 and a nudge management device 10. The nudge recommendation system 1 actually includes applications 101 of a plurality of communication terminals 100 of different users, but only the application 101 of one communication terminal 100 will be described herein for the purpose of convenience of explanation. The communication terminal 100 is a terminal that can perform wireless communication and examples thereof include a smartphone, a tablet terminal, and a PC terminal. The application 101 is an application that can be activated in the communication terminal 100 and an example thereof is a bus application for assisting with getting on a bus.

The nudge management device 10 is a device that manages information of a plurality of types of nudges and updates information of the nudges based on information from the application 101. The nudge management device 10 includes an acquisition unit 11, a storage unit 12, an abrasion deriving unit 13, a recovery deriving unit 14, and an update unit 15.

The acquisition unit 11 acquires various types of information from the application 101. For example, the acquisition unit 11 acquires nudge intervention information which is information associated with intervention of a nudge (information associated with intervention of a nudge in a user) for each of the plurality of types of nudges. The nudge intervention information may include, for example, whether there was a push notification on the previous day, whether there was a nudge intervention on the previous day, and a behavioral modification result of the previous day. The “previous day” in the following description means a previous day as an example of a predetermined period and may be a predetermined period (for example, last three days) other than the previous day. Here, whether there was a push notification is information indicating whether a push notification has been output by the communication terminal 100 of a user using the application 101. Whether there was a nudge intervention is information indicating whether a nudge on the nudge screen based on the push notification has been viewed by the user. The behavioral modification result is information indicating whether the user has adopted a behavior promoted by the nudge.

The acquisition unit 11 additionally acquires user information which is information associated with attributes of a user and which affects a decrease in effect of a nudge. The information associated with attributes of a user is information which is specific to the user based on personal features of the user. The user information may include, for example, usage times of the communication terminal 100 (smartphone) on the previous day, whether the user has gotten a COVID-19 vaccine, and a COVID-19 infection status of the previous day. Since how the communication terminal 100 of the user can be used can be estimated from the usage times of the communication terminal 100, for example, the user can be estimated to be a user who does not thoroughly check the screen or a user who sees through psychological effects of a nudge. Thoughts of the user about a risk can be estimated from the information indicating whether the user has gotten a COVID-19 vaccine. The current mental and physical conditions of the user or the like can be estimated from the COVID-19 infection status. The acquisition unit 11 may acquire whether the user has visited a racetrack, whether the user has visited tourist spots, the number of acquired points associated with the application 101, and the like as other user information. A degree of risk preference of the user can be estimated from information indicating whether the user has visited a racetrack. A sympathy bias or an intensity of need for approval can be estimated from information indicating whether the user has visited tourist spots. A bias for loss aversion or profits or a degree of participation in a campaign can be estimated from the number of acquired points. The aforementioned user information is either information that can affect a decrease in effect of nudges.

The acquisition unit 11 additionally acquires environment information indicating an external environment status of a user in a predetermined period. The external environment of a user is a factor around the user that can affect the user's behavior and a factor that is not associated with attributes of the user. The acquisition unit 11 may acquire the weather of the previous day, the temperature of the previous day, a day of the week of the previous day (whether the previous day is a weekday or Saturday or Sunday), and the number of COVID-19 infected people of the previous day. This information is information from which it can be estimated whether the user adopts a behavior of going out, close contact, or the like. The acquisition unit 11 may acquire factors that may affect a behavior of a user in addition to the aforementioned information. The acquisition unit 11 stores the acquired information in the storage unit 12.

The storage unit 12 is a database that stores information of a plurality of types of nudges and stores information acquired from the acquisition unit 11. The storage unit 12 stores, for example, information for uniquely identifying a nudge (a nudge ID), intervention conditions, details of a behavior to be promoted, a behavioral modification effect, an incentive, a behavioral modification means, and details of nudges in correlation with each other as the information of a plurality of types of nudges. The intervention conditions are information indicating in what case a nudge becomes a candidate for a nudge intervening and are defined, for example, as a “case in which a bus scheduled to get on is crowded.” The details of a behavior to be promoted are specific details of a behavior which a nudge prompts a user to adopt and are defined, for example, as “gets on an uncrowded next bus.” The behavioral modification effect is information indicating an extent (a probability) that a user adopts a behavior promoted by a nudge when nudge intervention is performed and is defined, for example, “10%.” The incentive is an incentive that is given to a user when the user adopts a behavior promoted by a nudge and is defined, for example, as “points 10p.” The behavioral modification means is information indicating by what means behavioral modification is performed such as a message, an image, or sound. The details of a nudge are information indicating specific details of the nudge (for example, details of a message (a wording of the nudge)). For example, a plurality of wordings such as “there is a rare uncrowded bus after the next one!” and “avoidance of crowdedness causes contribution to the society” are defined.

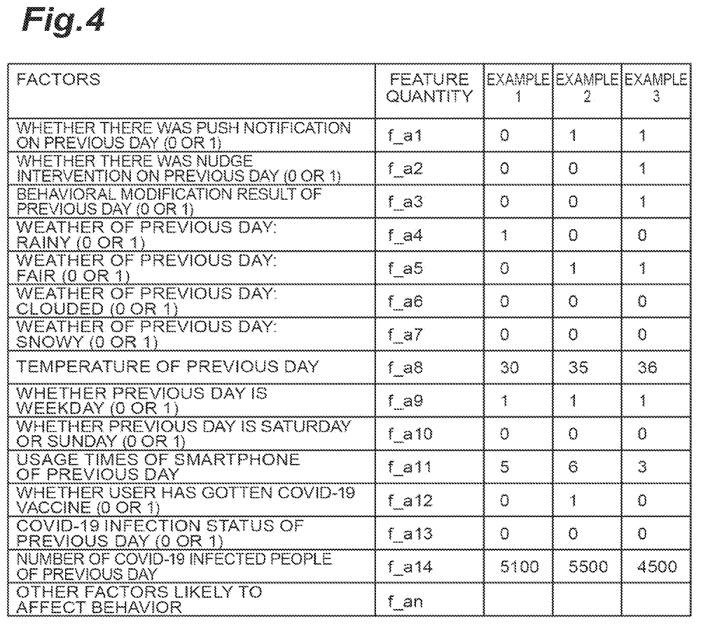

As information acquired from the acquisition unit 11, for example, as illustrated in FIG. 4, the storage unit 12 stores a feature quantity f_a1 representing whether there was a push notification on the previous day using 0 or 1, a feature quantity f_a2 representing whether there was a nudge intervention on the previous day using 0 or 1, a feature quantity f_a3 representing a behavioral modification result of the previous day using 0 or 1, a feature quantity f_a4 representing whether the weather of the previous day is rainy using 0 or 1, a feature quantity f_a5 representing whether the weather of the previous day was fair using 0 or 1, a feature quantity f_a6 representing whether the weather of the previous day was cloudy using 0 or 1, a feature quantity f_a7 representing whether the weather of the previous day was snowy using 0 or 1, a feature quantity f_a8 representing the temperature of the previous day, a feature quantity f_a9 representing whether the previous day was a weekday using 0 or 1, a feature quantity f_a10 representing whether the previous day was Saturday or Sunday using 0 or 1, a feature quantity f_a11 representing usage times of a smartphone of the previous day, a feature quantity f_a12 representing whether a user has gotten a COVID-19 vaccine using 0 or 1, a feature quantity f_a13 representing a COVID-19 infection status of the previous day using 0 or 1, a feature quantity f_a14 representing the number of COVID-19 infected people of the previous day, and a feature quantity f an representing other factors that can affect a user's behavior. Feature quantities of Examples 1 to 3 acquired by the acquisition unit 11 at different timings are illustrated in FIG. 4.

The abrasion deriving unit 13 is configured to derive nudge abrasion information indicating a degree of decrease in effect of a nudge associated with the nudge intervention information based on the nudge intervention information and to output the derived nudge abrasion information. The nudge intervention information is information including, for example, whether there was a push notification on the previous day, whether there was a nudge intervention on the previous day, and a behavioral modification effect of the previous day. The abrasion deriving unit 13 may derive nudge abrasion information for each of a plurality of types of nudges. The abrasion deriving unit 13 identifies feature quantities associated with the nudge intervention information with reference to the information stored in the storage unit 12 and derives the nudge abrasion information based on the values of the identified feature quantities. In the example illustrated in FIG. 4, the feature quantity f_a1 representing whether there was a push notification on the previous day is set to 1 when there was a notification and set to 0 when there was no notification. The feature quantity f_a2 representing whether there was a nudge intervention on the previous day is set to 1 when there was an intervention and set to 0 when there was no intervention. The feature quantity f_a3 representing the behavioral modification result of the previous day is set to 1 when behavioral modification has been achieved (when a user has adopted a behavior promoted by a nudge) and set to 0 when behavioral modification has not been achieved (when a user has not adopted a behavior promoted by a nudge).

When there was no push notification on the previous day (f_a1=0) or when there was no nudge intervention on the previous day (f_a2=0), the abrasion deriving unit 13 sets abrasion(date) indicating abrasion to abrasion(date)=0 and derives nudge abrasion information indicating that no abrasion occurs. This is because it can be determined that a nudge has not been abraded because of no nudge intervention. For example, in Examples 1 and 2 illustrated in FIG. 4, nudge abrasion information indicating that no abrasion occurs is derived.

On the other hand, when there was a push notification on the previous day (f_a1=1) and there was a nudge intervention on the previous day (f_a2=1), the abrasion deriving unit 13 derives nudge abrasion information in additional consideration of user information and environment information. The user information is, for example, information including usage times of a smartphone of the previous day, whether a user has gotten a COVID-19 vaccine, and a COVID-19 infection status of the previous day as described above. In the example illustrated in FIG. 4, the feature quantity f_a11 representing usage times of a smartphone of the previous day has a value in a range of 0 to 24 hours. The feature quantity f_a12 representing whether a user has gotten a COVID-19 vaccine is set to 1 when the user has gotten a COVID-19 vaccine and is set to 0 when the user has not gotten a COVID-19 vaccine. The feature quantity f_a13 representing a COVID-19 infection status of the previous day is set to 1 when the user has been infected and is set to 0 when the user has not been infected.

As described above, the environment information is, for example, information including the weather of the previous day, the temperature of the previous day, the day of the previous day (whether the previous day was a weekday or Saturday or Sunday), and the number of COVID-19 infected people of the previous day. In the example illustrated in FIG. 4, the feature quantity f_a4 representing whether the weather of the previous day was rainy is set to 1 when the weather is rainy and set to 0 when the weather is not rainy. The feature quantity f_a5 representing whether the weather of the previous day was fair is set to 1 when the weather is fair and set to 0 when the weather is not fair. The feature quantity f_a6 representing whether the weather of the previous day was cloudy is set to 1 when the weather is cloudy and set to 0 when the weather is not cloudy. The feature quantity f_a7 representing whether the weather of the previous day was snowy is set to 1 when the weather is snowy and set to 0 when the weather is not snowy. The feature quantity f_a8 representing the temperature of the previous day is set to a value indicating the temperature. The feature quantity f_a9 representing whether the previous day is a weekday is set to 1 when the previous day is a weekday and set to 0 when the previous day is not a weekday. The feature quantity f_a10 representing whether the previous day is Saturday or Sunday is set to 1 when the previous day is Saturday or Sunday and set to 0 when the previous day is not Saturday or Sunday. The feature quantity f_a14 representing the number of COVID-19 infected people of the previous day is set to a value indicating the number of infected people.

When there was a push notification on the previous day (f_a1=1) and there was a nudge intervention on the previous day (f_a2=1), the abrasion deriving unit 13 derives abrasion(date) indicating abrasion using Expression (1) in consideration of the user information and the environment information. In the following expression, wα is a weight and has a value varying according to a model case. Here, abrasion(date) indicating abrasion is adjusted to have a value in a range of 0 to 1.

$$\begin{matrix}[ Math . 1 ] & \\ abrasion ( date ) = {\sum }_{i = 3}^{n} {w}_{i}^{α} f\_ai & ( 1 )\end{matrix}$$

For example, wα indicating a weight may be derived by solving a regressive equation using a least square method. For example, wα indicating a weight may be derived from a correlation coefficient between each feature quantity (column) and a behavioral modification result which is an objective variable. For example, wα indicating a weight may be derived by preparing a machine learning model for predicting the behavioral modification result which is an objective variable and using a contribution ratio of each feature quantity (column).

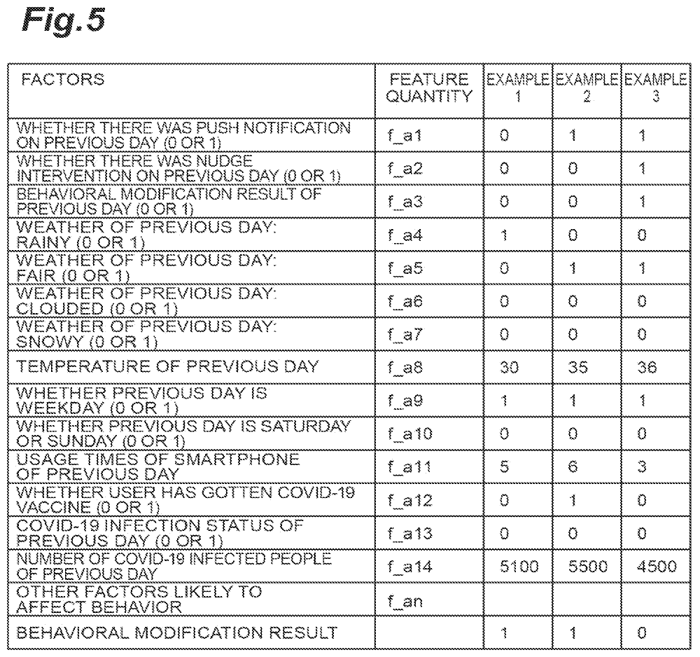

When the nudge abrasion information is derived using Expression (1), a behavioral modification result in consideration of the nudge abrasion information may be derived for each of the examples (Examples 1 to 3) as illustrated in FIG. 5, for example, using the machine learning model for predicting the behavioral modification result which is an objective variable. In the example illustrated in FIG. 5, the behavioral modification result is set to 1 when it is predicted that behavioral modification has been achieved (when it is predicted that a user adopts a behavior promoted by a nudge) and set to 0 when it is predicted that behavioral modification has not been achieved (when it is predicted that a user does not adopt a behavior promoted by a nudge).

When there was a push notification on the previous day (f_a1=1) and there was a nudge intervention on the previous day (f_a2=1), it has been described above that the abrasion deriving unit 13 derives abrasion(date) indicating abrasion using Expression (1), but the present invention is not limited thereto, and abrasion information may be derived as follows. An example in which abrasion information is derived from whether there was a push notification on the previous day (f1), whether there was a nudge intervention on the previous day (f2), and a behavioral modification effect (f3) of the previous day which are the nudge intervention information and a visit frequency (f4) to a certain tourist spot which is the user information will be described below. For example, the visit frequency f4 is 10 when the value is greater than 10 and is set to a value obtained by dividing the value by 10. It is assumed that weights of f1 to f4 in deriving abrasion information are 0.1, 0.2, 0.6, and 0.1. In this case, when f1=1, f2=1, f3=1, and f4=0.2 are assumed, the abrasion is calculated as 0.1×1+0.2×1+0.6×1+0.1×0.2=0.92 in consideration of the weights. When f1=1, f2=1, f3=0, and f4=0.2 are assumed, the abrasion is calculated as 0.1×1+0.2×1+0.6×0+0.1×0.2=0.32.

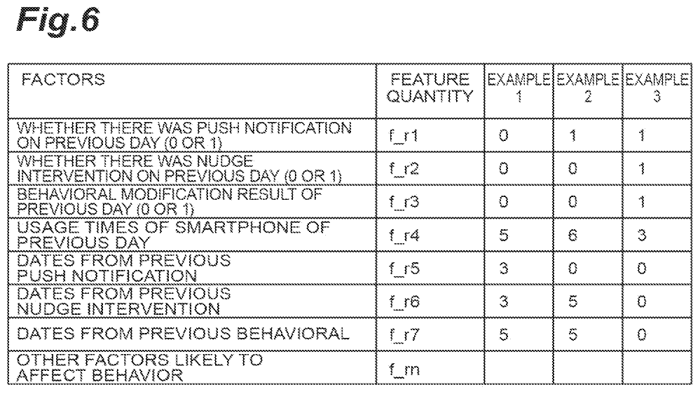

The recovery deriving unit 14 is configured to derive nudge recovery information indicating a degree of recovery in effect of a nudge associated with the nudge intervention information based on the nudge intervention information and to output the derived nudge recovery information. The nudge recovery information is information indicating a degree of recovery of a nudge which has been temporarily abraded. As described above, the nudge intervention information is information including, for example, whether there was a push notification on the previous day, whether there was a nudge intervention on the previous day, and a behavioral modification effect of the previous day. The recovery deriving unit 14 may derive nudge recovery information for each of a plurality of types of nudges. The recovery deriving unit 14 identifies feature quantities associated with the nudge intervention information with reference to the information stored in the storage unit 12 and derives the nudge recovery information based on the values of the identified feature quantities. In the example illustrated in FIG. 6, a feature quantity f_r1 representing whether there was a push notification on the previous day is set to 1 when there was a notification and set to 0 when there was no notification. A feature quantity f_r2 representing whether there was a nudge intervention on the previous day is set to 1 when there was an intervention and set to 0 when there was no intervention. The feature quantity f_r3 representing the behavioral modification result of the previous day is set to 1 when behavioral modification has been achieved (when a user has adopted a behavior promoted by a nudge) and set to 0 when behavioral modification has not been achieved (when a user has not adopted a behavior promoted by a nudge).

When behavioral modification has been achieved on the previous day (f_r3=1), the recovery deriving unit 14 sets recovery(date) indicating recovery to recovery(date)=0 and derives nudge recovery information indicating that the nudge has not been recovered. This is because it can be determined that there is a high likelihood that a nudge has been abraded and the nudge has not been recovered when a user has adopted the behavioral modification using the nudge. For example, in Example 3 illustrated in FIG. 6, nudge recovery information indicating that the nudge has not been recovered is derived.

On the other hand, when behavioral modification has not been achieved on the previous day (f_r3=0), the recovery deriving unit 14 derives the nudge recovery information in additional consideration of other information. In the example illustrated in FIG. 6, usage times of a smartphone of the previous day, dates from the last push notification, dates from the last nudge intervention, dates from the last behavioral modification, and factors which may affect a behavior are illustrated as the other information.

When behavioral modification has not been achieved on the previous day (f_r3=0), the recovery deriving unit 14 derives recovery(date) indicating recovery using Expression (2) in consideration of the aforementioned other information. In the following expression, wr is a weight and has a value varying according to a model case. Here, wr can be calculated, for example, in the same way as wα indicating a weight associated with derivation of abrasion. In addition, recovery(date) indicating recovery is adjusted to have a value in a range of 0 to 1.

$$\begin{matrix}[ Math . 2 ] & \\ recovery ( date ) = {\sum }_{i = 1}^{n} {w}_{i}^{r} f\_ri & ( 2 )\end{matrix}$$

The update unit 15 is configured to update the information of nudges stored in the storage unit 12 based on the nudge abrasion information and the nudge recovery information. The update unit 15 acquires nudge abrasion information of each nudge from the abrasion deriving unit 13. The update unit 15 acquires nudge recovery information of each nudge from the recovery deriving unit 14. The update unit 15 acquires abrasion information at the current time point of each nudge from the information of nudges stored in the storage unit 12. The update unit 15 derives newest information of abrasion based on the nudge abrasion information, the nudge recovery information, and the current information of abrasion for each nudge and updates the information of each nudge stored in the storage unit 12 based on the derived information of abrasion.

The update unit 15 may update the information of one nudge included in a plurality of types of nudges in consideration of both nudge abrasion information of the one nudge and nudge abrasion information of nudges similar to the one nudge.

For example, abrasion of a nudge is expressed by following recurrence formulas of Expressions (3) and (4).

$$\begin{matrix}[ Math . 3 ] & \\ {F}_{i}^{( 0 )} = 0 & ( 3 )\end{matrix}$$

$$\begin{matrix}[ Math . 4 ] & \\ {F}_{I}^{( N )} = {\sum }_{J = 1}^{J = nudge \_ num} {W}_{I \_ J} \{ {abrasion}_{I} ( date ) - {recovery}_{I} ( date ) + {F}_{I}^{( N - 1 )} \} & ( 4 )\end{matrix}$$

Expression (3) represents that the nudge has not been abraded when N=0. Expression (4) represents that, when abrasion F(N)I of one nudge is derived, a value for each nudge is derived by subtracting recovery(date) indicating recovery from abrasion(date) indicating abrasion, adding F(N=1)I which is information of abrasion at a current time point to the resultant value, and multiplying the resultant value by the weight WI_J, and the abrasion FNI of the one nudge is derived by adding the values of the nudges. The weight WI_J for a nudge not similar to the one nudge is 0. Accordingly, abrasion of a nudge is derived in consideration of only information of nudges similar to each other.

For example, it is assumed that the plurality of types of nudges include a nudge A, a nudge B, and nudge C which are similar to each other. In this case, abrasion F(N)A of the nudge A is derived by Expression (5). In Expression (5), WA_A is a weight of the nudge A when abrasion of the nudge A is represented, WA_B is a weight associated with derivation of abrasion of the nudge B when abrasion of the nudge A is represented, and WA_C is a weight associated with derivation of abrasion of the nudge C when abrasion of the nudge A is represented. WA_B (or WA_C) is set based on a degree of similarity between the nudge A and the nudge B (or the nudge C).

$$\begin{matrix}[ Math . 5 ] & \\ {F}_{A}^{( N )} = \text{?} \{ {abrasion}_{A} ( date ) - {recovery}_{A} ( date ) + {F}_{A}^{( N - 1 )} \} + \text{?} \{ {abrasion}_{B} ( date ) - {recovery}_{B} ( date ) + \text{?} \} + \text{?} \{ {abrasion}_{C} ( date ) - {recovery}_{C} ( date ) + \text{?} \} & ( 5 )\end{matrix}$$

$$\text{?} \text{indicates text missing or illegible when filed}$$

A weight WI_J will be described below in detail. When abrasion of one nudge is derived from abrasion of a plurality of types of nudges, the weight of abrasion of the one nudge (a target nudge) is the largest. The weight of abrasion of a nudge with a higher degree of similarity to the target nudge is larger. A high degree of similarity means that a degree of overlap of cognitive biases (psychological biases) associated with a nudge is high. For example, it is assumed that biases a, b, and c are associated with the nudge A, biases b, c, and d are associated with the nudge B, and a bias d is associated with the nudge C. In this case, a degree of similarity corresponding to a degree of overlap of biases may be derived, for example, based on |number of biases common to target bias/number of unique biases of two nudges].

In the aforementioned example, it is assumed that the nudge A is a target nudge. In this case, since the number of biases common to the target nudge (itself) is 3 and the number of unique biases in two nudges is 3, WA_A is 3/3=1.0. Since the number of biases b and c common to the target nudge is 2 and the number of unique biases a, b, c, and d in two nudges is 4, WA_B is 2/4=0.5. Since the number of biases common to the target nudge is 0 and the number of unique biases a, b, c, and d in two nudges is 4, WA_B is 0/4=0. When the number of common biases is 0, it means that two nudges are not similar to each other.

Similarly, it is assumed that the nudge B is a target nudge. In this case, since the number of biases b and c common to the target nudge is 2 and the number of unique biases a, b, c, and d in two nudges is 4, WB_A associated with derivation of abrasion of the nudge A when abrasion of the nudge B is represented is 2/4=0.5. WB_B is 1.0. Since the number of biases d common to the target nudge is 1 and the number of unique biases b, c, and d in two nudges is 3, the weight WB_C associated with derivation of abrasion of the nudge A when abrasion of the nudge B is represented is 1/3=0.333 . . . .

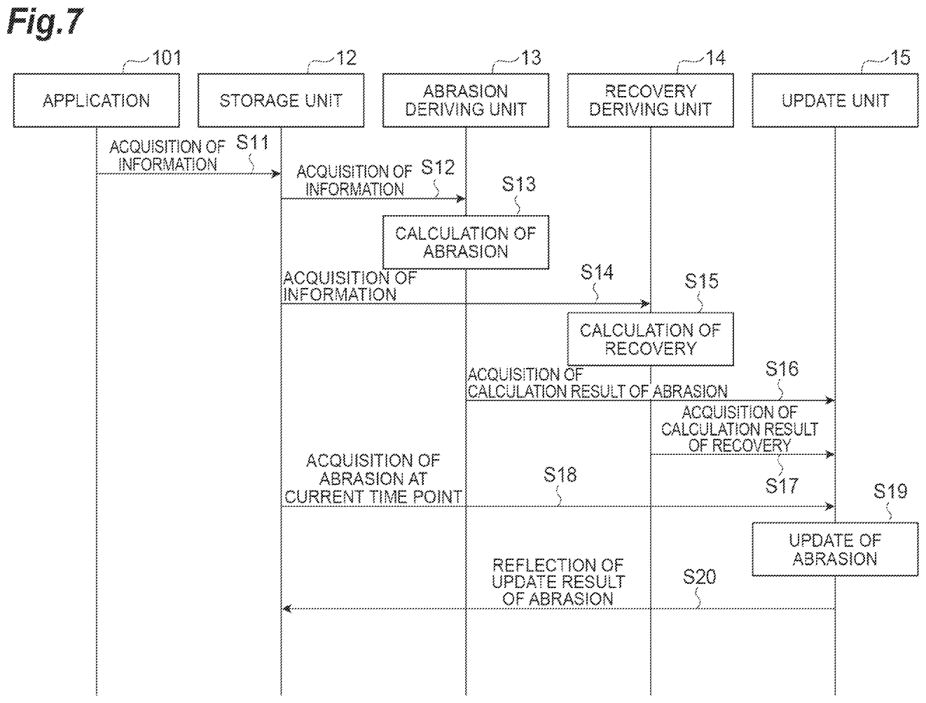

A nudge abrasion updating process will be described below with reference to FIG. 7. FIG. 7 is a sequence diagram illustrating a nudge abrasion updating process.

As illustrated in FIG. 7, various types of information including nudge intervention information are acquired from the application 101 by the acquisition unit 11 and are stored in the storage unit 12 (Step S11).

Then, information (information including nudge intervention information) stored in the storage unit 12 is acquired by the abrasion deriving unit 13 (Step S12). Then, nudge abrasion information of each nudge is derived based on the nudge intervention information or the like by the abrasion deriving unit 13 (Step S13).

In addition, information (information including nudge intervention information) stored in the storage unit 12 is acquired by the recovery deriving unit 14 (Step S14). Then, nudge recovery information of each nudge is derived based on the nudge intervention information or the like by the recovery deriving unit 14 (Step S15).

Subsequently, a result of derivation of nudge abrasion information from the abrasion deriving unit 13 is acquired by the update unit 15 (Step S16), and a result of derivation of nudge recovery information from the recovery deriving unit 14 is acquired by the update unit 15 (Step S17). Abrasion information at a current time point of each nudge is acquired from the storage unit 12 by the update unit 15 (Step S18).

Then, an abrasion updating process is performed based on the acquired information by the update unit 15 (Step S19), and a result of abrasion update is reflected in the information of each nudge stored in the storage unit 12 (Step S20).

Operations and advantages of the nudge management device 10 according to the first embodiment will be described below.

The nudge management device 10 according to this embodiment includes: a storage unit 12 configured to store information of a nudge which is a mechanism for prompting a user to voluntarily adopt a desirable behavior; an acquisition unit 11 configured to acquire nudge intervention information which is information associated with intervention of a nudge (information associated with intervention of a nudge in a user); and an abrasion deriving unit 13 configured to derive nudge abrasion information indicating a degree of decrease in effect of the nudge associated with the nudge intervention information based on the nudge intervention information and to output the derived nudge abrasion information.

In the nudge management device 10 according to this embodiment, nudge intervention information which is information associated with intervention of a nudge (information associated with a nudge's intervention in a user) is acquired, and nudge abrasion information indicating a degree of decrease in effect of the nudge is derived and output based on the nudge intervention information. In this way, since the nudge abrasion information is estimated (derived) from the information associated with a nudge's intervention in a user (for example, a nudge intervention frequency), it is possible to appropriately (accurately) represent abrasion of the nudge using a numerical value, for example, when the nudge is abraded (a behavioral modification effect of the nudge decreases) due to repeated intervention of the nudge. As described above, with the nudge management device 10 according to this embodiment, it is possible to appropriately represent abrasion of a nudge using a numerical value.

By appropriately representing abrasion of a nudge using a numerical value, for example, the following merits are obtained. Firstly, when a nudge is recommended to a user, recommendation in consideration of abrasion of the nudge is possible. Recommendation can be performed based on magnitude relationship between numerical values or the numerical value of abrasion can be used as a feature quantity of machine learning associated with the recommendation. Recommendation in consideration of abrasion will be described later in detail in a second embodiment. Secondly, it is possible to identify a nudge which is generally likely to be abraded. By calculating an average or the like of abrasion of nudges at a time point at which a certain amount of data has been collected, it is possible to identify a nudge which is likely to be abraded and a nudge which is less likely to be abraded and to examine a new nudge based on cognitive biases associated with the nudges. Thirdly, it is possible to identify an upper limit of abrasion. Here, the upper limit of abrasion means a value at which abrasion does not progress any more or which is not effectual any more even when the numerical value of abrasion increases apparently. By identifying the upper limit of abrasion in a scene of use, it is possible to ascertain that further continuous application of a nudge is not meaningful any more. Fourthly, it is possible to improve accuracy of inspection of recovery of a nudge or collection of data. When inspection of recovery of a nudge or collection of data under specific conditions is performed, it is conceivable that the nudge be intentionally abraded and intervention of the nudge be performed under the same conditions. In this case, by representing abrasion using a numerical value, it is possible to appropriately perform inspection of recovery of a nudge or collection of data according to conditions.

In returning to the description of operations and advantages, the nudge intervention information may include at least information indicating whether the nudge has been viewed by the user in a predetermined period and information indicating whether the user has adopted a behavior promoted by the nudge. With this configuration, since it is identified whether the nudge is a nudge having intervened in the user (viewed by the user) and whether the nudge has actually modified the user's behavior, it is possible to more appropriately derive abrasion of the nudge based on information closely associated with the abrasion of the nudge.

The acquisition unit 11 may additionally acquire user information which is associated with attributes of the user and which affects the decrease in effect of the nudge, and the abrasion deriving unit 13 may derive the nudge abrasion information in additional consideration of the user information. With this configuration, since the nudge abrasion information is derived in additional consideration of information which is associated with attributes of a user such as usage times of a smartphone or whether the user has visited a predetermined place and which can affect a decrease in effect of a nudge, it is possible to more appropriately derive abrasion of a nudge.

The acquisition unit 11 may additionally acquire environment information indicating an external environment state of the user in a predetermined period, and the abrasion deriving unit 13 may derive the nudge abrasion n information in additional consideration of the environment information. With this configuration, since the nudge abrasion information is derived in additional consideration of environment information indicating an external environment status of the user such as the weather or the number of COVID-19 infected people, it is possible to more appropriately derive abrasion of a nudge.

The nudge management device 10 may further include an update unit 15 configured to update information of the nudge associated with the nudge intervention information and stored in the storage unit 12 based on the nudge abrasion information. In this way, by using the nudge abrasion information derived as a numerical value to update information of nudges in the storage unit 12, it is possible to reflect the nudge abrasion information in original information of nudges.

The storage unit 12 may store information of a plurality of types of nudges, the abrasion deriving unit 13 may derive the nudge abrasion information for each of the plurality of types of nudges, and the update unit 15 may update information of one of the nudges in consideration of both the nudge abrasion information of the one nudge included in the plurality of types of nudge and the nudge abrasion information of nudges similar to the one nudge. For example, when a nudge similar to one nudge repeatedly intervenes in a user, the one nudge is considered to be abraded with an influence thereof. In this regard, by updating information of one nudge in consideration of nudge abrasion information of a nudge similar to the one nudge in addition to nudge abrasion information of the one nudge, it is possible to update the information of the one nudge based on an abrasion status more based on an actual state of the one nudge.

The nudge management device 10 may further include a recovery deriving unit 14 configured to derive nudge recovery information indicating a degree of recovery in effect of the nudge associated with the nudge intervention information based on the nudge intervention information, and the update unit 15 may update information of the nudge associated with the nudge intervention information in additional consideration of the nudge recovery information. For example, when a nudge having repeatedly intervened in a user and having been abraded once does not intervene in the user in a predetermined period, or the like, the effect of the nudge is considered to be recovered. Accordingly, by deriving nudge recovery information based on the nudge intervention information and updating information of a nudge in consideration of the nudge recovery information, it is possible to update information of a nudge with information more based on an actual state of the nudge.

### Second Embodiment

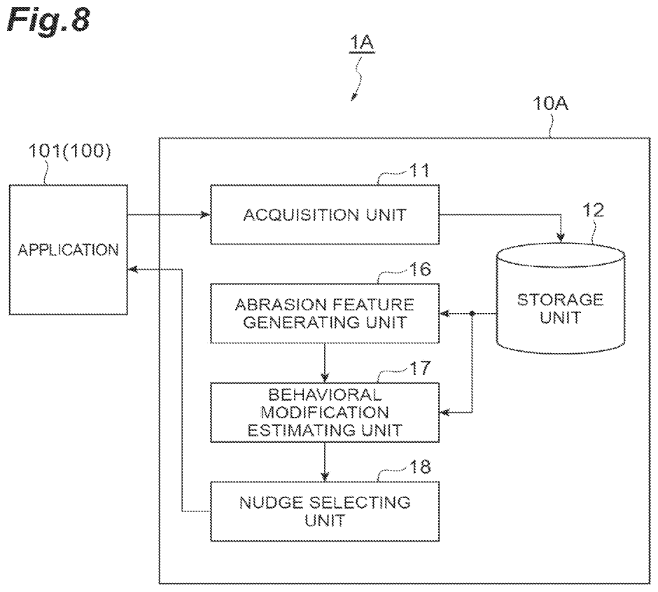

A nudge management device 10A (an information processing device) included in a nudge recommendation system 1A according to a second embodiment will be described below with reference to FIGS. 8 to 12. In the second embodiment, description common to the first embodiment will be omitted and differences from the first embodiment will be mainly described.

FIG. 8 is a functional block diagram of the nudge management device 10A included in the nudge recommendation system 1A according to the second embodiment. The nudge management device 10A has a function of recommending a nudge with a high behavioral modification probability which is a probability that a user will adopt a behavior promoted by the nudge to the user in consideration of abrasion of the nudge.

As illustrated in FIG. 8, the nudge management device 10A includes an acquisition unit 11, a storage unit 12, an abrasion feature generating unit 16 (an abrasion deriving unit), a behavioral modification estimating unit 17, and a nudge selecting unit 18.

The acquisition unit 11 acquires various types of information in correlation with each other from the application 101 when specific conditions are satisfied. The specific conditions means specific conditions in which a nudge can be recommended to a user, and a specific example thereof is to check in at a specific spot (such as a store). The acquisition unit 11 acquires, for example, nudge intervention information. The nudge intervention information in this case may include, for example, whether there was a push notification on the previous day, an intervention-scheduled nudge, whether there was a nudge intervention on the previous day, and a behavioral modification result of the previous day. The intervention-scheduled nudge is a nudge which is scheduled to intervene in a user (or which intervenes actually in a user) when the user ascertains the push notification.

The acquisition unit 11 may acquire user information which is information associated with attributes of a user as information included in the various types of information. The user information includes, for example, a user ID for uniquely identifying the user, sex, age, and family members. The acquisition unit 11 may acquire the weather, the temperature, the day, usage times of a smartphone by a user, whether a user has gotten a COVID-19 vaccine, a COVID-19 infection status of a user, the number of COVID-19 infected people of the previous day, and a close-contact flag of a user as other information (feature quantities likely to contribute to a behavior) included in the various types of information. The acquisition unit 11 stores the acquired information in the storage unit 12.

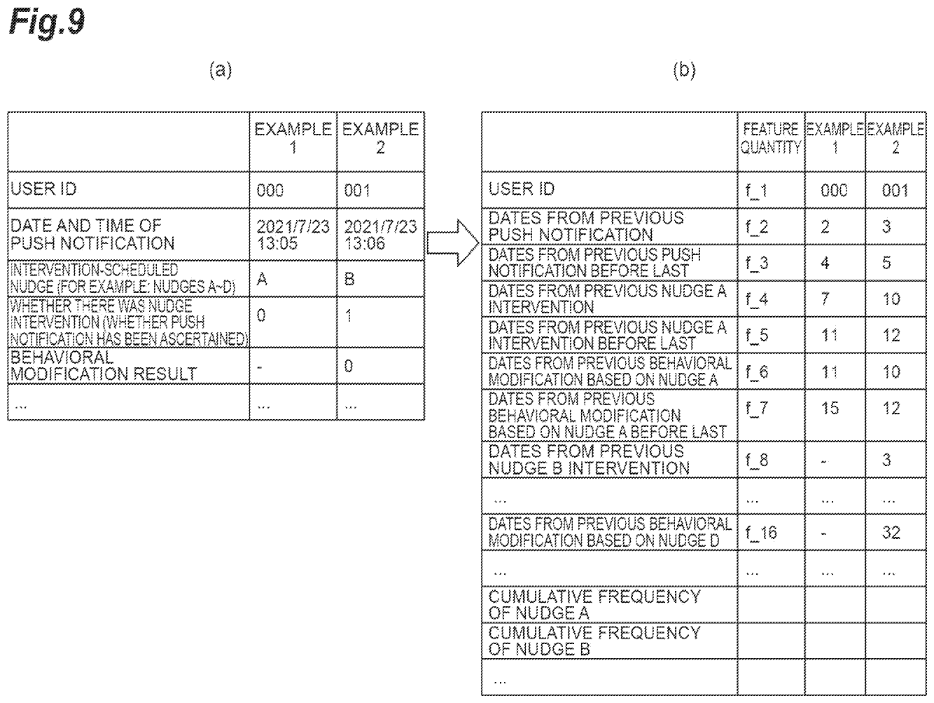

The abrasion feature generating unit 16 generates an abrasion feature quantity representing nudge abrasion information based on the nudge intervention information for each of a plurality of types of nudges. FIG. 9 is a diagram illustrating generation of an abrasion feature quantity. FIG. 9(a) illustrates information (a log) including nudge intervention information which is acquired by the acquisition unit 11 and stored in the storage unit 12. Records of this log are generated whenever a nudge is presented through a push notification. The abrasion feature generating unit 16 converts the log to an abrasion feature quantity illustrated in FIG. 9(b).

In the example illustrated in FIG. 9(b), a feature quantity f_1 indicating a user ID, a feature quantity f_2 indicating dates from a previous push notification, a feature quantity f_3 indicating dates from a previous push notification before last, a feature quantity f_4 indicating dates from a previous nudge A intervention, a feature quantity f_5 indicating dates from a previous nudge A intervention before last, a feature quantity f_6 indicating dates from previous behavioral modification by the nudge A, a feature quantity f_7 indicating dates from previous behavioral modification by the nudge A before last, a feature quantity f_8 indicating dates from a previous nudge B intervention, . . . , and a feature quantity f_16 indicating dates from previous behavioral modification by the nudge D are illustrated. Although not illustrated in FIG. 9(b), the same feature quantities as the nudge A are defined for each nudge. As illustrated in FIG. 9(b), cumulative frequencies of each nudge may be defined as feature quantities.

The behavioral modification estimating unit 17 estimates a behavioral modification probability which is a probability that a user will adopt a behavior promoted by a nudge. The behavioral modification estimating unit 17 estimates the behavioral modification probability for each of a plurality of types of nudges based on the abrasion feature quantities of the plurality of types of nudges.

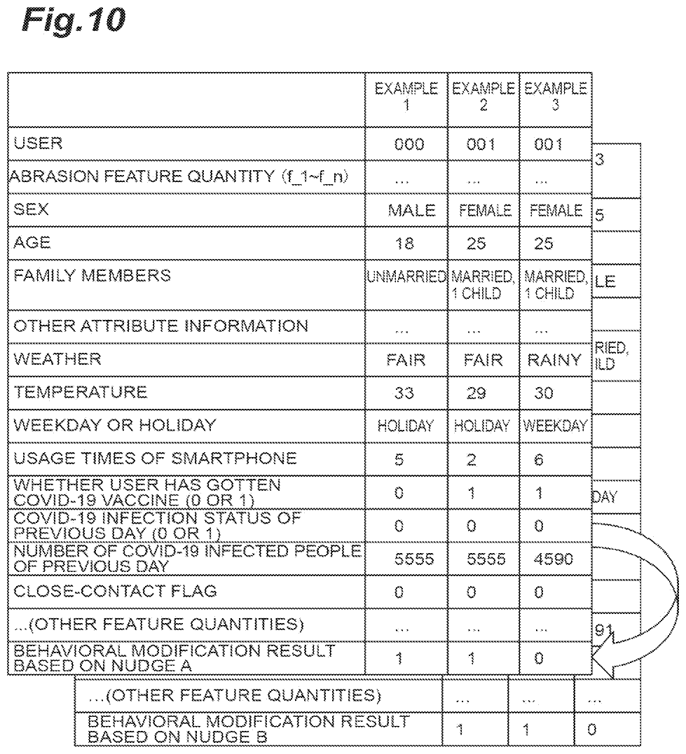

The behavioral modification estimating unit 17 generates an estimation model (a machine learning model) for estimating a behavioral modification probability for each of a plurality of types of nudges based on an abrasion feature quantity and user information. FIG. 10 is a diagram illustrating estimation of behavioral modification and specifically a diagram illustrating generation of an estimation model for estimating a behavioral modification probability. In the example illustrated in FIG. 10, the behavioral modification estimating unit 17 generates an estimation model for estimating a behavioral modification probability of a nudge A using the abrasion feature quantity, a user ID, sex, age, and family members which are the user information, the weather, the temperature, the day, usage times of a smartphone by a user, whether a user has gotten a COVID-19 vaccine, a COVID-19 infection status of a user, the number of COVID-19 infected people of the previous day, and a close-contact flag of a user which are the other information (feature quantities likely to contribute to a behavior), and a behavioral modification result (an objective variable) as training data. As training data including a plurality of records are present in Examples 1 to 3 of FIG. 10, a plurality of pieces of training data may be present for the same nudge due to differences in information acquisition timings (differences in a nudge intervention scene) even for the same user.

The behavioral modification estimating unit 17 estimates a behavioral modification probability for each of a plurality of types of nudges based on the estimation model. The behavioral modification estimating unit 17 estimates the behavioral modification probability of a target user for each nudge by inputting data such as the aforementioned training data to the estimation model for each nudge.

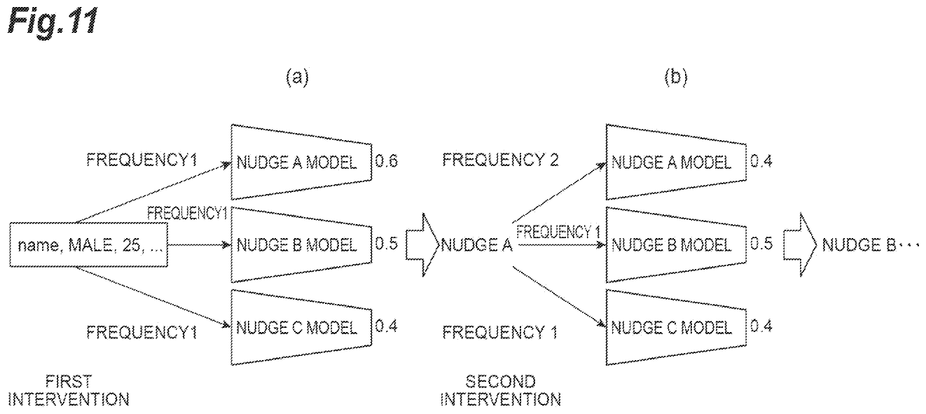

The nudge selecting unit 18 selects a nudge to be recommended to the target user based on the behavioral modification probability. Specifically, the nudge selecting unit 18 selects a nudge with a highest behavioral modification probability as the nudge to be recommended to the user. FIG. 11 is a diagram illustrating selection of a nudge. An example in which an optimal nudge is selected out of nudges A to C will be described now. It is assumed that estimation models for the nudges A to C (a nudge A model, a nudge B model, and a nudge C model) are generated for a target user (male, 25 years). As illustrated in FIG. 11(a), it is assumed that the behavioral modification probability estimated using the nudge A model in the first nudge intervention is 0.6, the behavioral modification probability estimated using the nudge B model is 0.5, and the behavioral modification probability estimated using the nudge C model is 0.4. In this case, the nudge selecting unit 18 selects the nudge A with the highest behavioral modification probability as a nudge to be recommended. When the nudge A is recommended at the last, the behavioral modification probability of the nudge A estimated using the nudge A model can be decreased. Accordingly, as illustrated in FIG. 11(b), it is assumed that the behavioral modification probability estimated using the nudge A model in the second nudge intervention is 0.4, the behavioral modification probability estimated using the nudge B model is 0.5, and the behavioral modification probability estimated using the nudge C model is 0.4. In this case, the nudge selecting unit 18 selects the nudge B with the highest behavioral modification probability as a nudge to be recommended. In this way, the behavioral modification probability is updated based on the nudge intervention information, and an optimal nudge is selected.

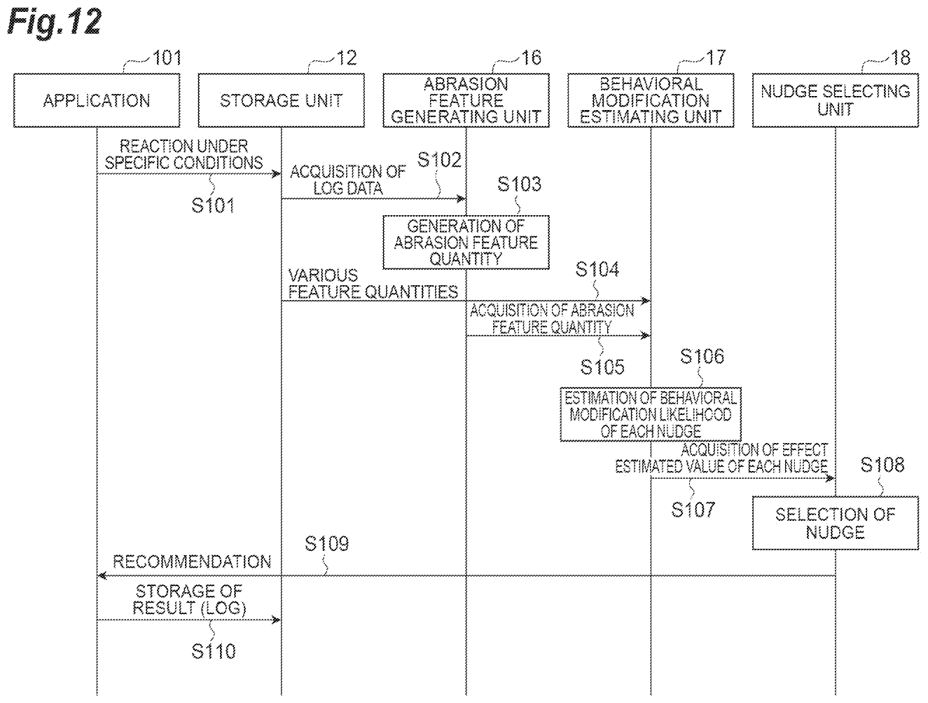

A nudge recommending process will be described below with reference to FIG. 12. FIG. 12 is a sequence diagram illustrating the nudge recommending process.

As illustrated in FIG. 12, when specific conditions are satisfied, various types of information including the nudge intervention information are acquired from the application 101 by the acquisition unit 11 and stored in the storage unit 12 (Step S101).

Then, the abrasion feature generating unit 16 acquires log data including the nudge intervention information stored in the storage unit 12 (Step S102) and generates an abrasion feature quantity of each nudge based on the log data (Step S103).

Then, the behavioral modification estimating unit 17 acquires various feature quantities such as user information stored in the storage unit 12 (Step S104), acquires the abrasion feature quantity of each nudge from the abrasion feature generating unit 16 (Step S105), and estimates a behavioral modification probability (a likelihood of behavioral modification) of each nudge based on the abrasion feature quantities, the user information, and the like (Step S106).

Subsequently, the nudge selecting unit 18 acquires the behavioral modification probability (an effect estimated value) of each nudge and selects a nudge to be recommended to a target user out of a plurality of nudges based on the behavioral modification probabilities (Step S108). Then, the selected nudge is recommended to the target user via the application 101 (Step S109). The nudge intervention information of the recommended nudge is stored in the storage unit 12 via the acquisition unit 11 (Step S110) and is used as log data to generate an abrasion feature quantity later.

Operations and advantages of the nudge management device 10A according to the second embodiment will be described below.

The nudge management device 10A according to this embodiment may further include: a behavioral modification estimating unit 17 configured to estimate a behavioral modification probability which is a probability with which the user adopts a behavior promoted by the nudge; and a nudge selecting unit 18 configured to select a nudge to be recommended to a user based on the behavioral modification probability, the storage unit 12 may store information of the plurality of types of nudges, the abrasion deriving unit may include an abrasion feature generating unit 16 configured to generate an abrasion feature quantity indicating the nudge abrasion information based on the nudge intervention information for each of the plurality of types of nudges, the behavioral modification estimating unit 17 may estimate the behavioral modification probability for each of the plurality of types of nudges based on the abrasion feature quantities of the plurality of types of nudges, and the nudge selecting unit 18 may select a nudge with the highest behavioral modification probability as the nudge to be recommended to the user.

In this way, since the behavioral modification probability for each of the plurality of types of nudges is estimated based on the abrasion feature quantity representing the nudge abrasion information and a nudge with a high behavioral modification probability is selected as a nudge to be recommended, it is possible to more accurately estimate the behavioral modification probability in consideration of the nudge abrasion information and to appropriately select a nudge that can promote behavioral modification of a user.

The behavioral modification estimating unit 17 may generate an estimation model for estimating the behavioral modification probability for each of the plurality of types of nudges based on the abrasion feature quantities and user information which is information associated with attributes of a user and estimate the behavioral modification probability for each of the plurality of types of nudges based on the estimation model. In this way, since an estimation model is generated for each nudge in consideration of the abrasion feature quantity and the user information and the behavioral modification probability for each nudge is estimated based on the estimation model, it is possible to more simply and accurately estimate the behavioral modification probability of each nudge.

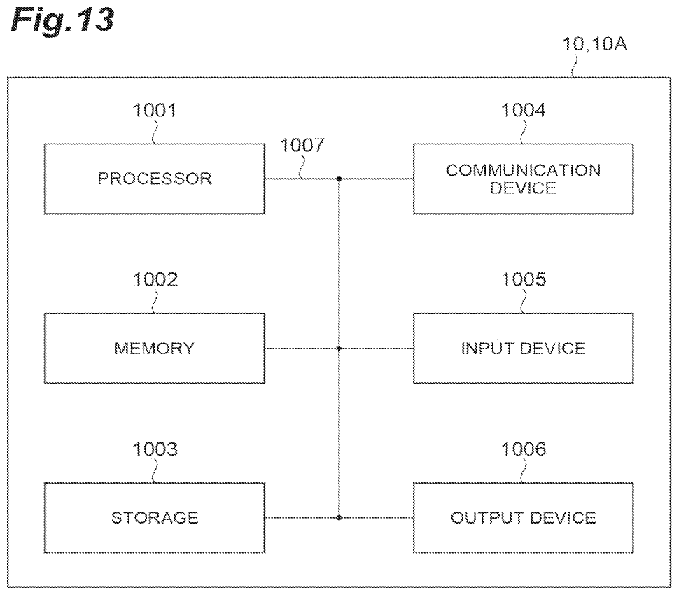

A hardware configuration of the nudge management device 10 or 10A will be described below with reference to FIG. 13. The nudge management device 10 or 10A may be physically configured as a computer device including a processor 1001, a memory 1002, a storage 1003, a communication device 1004, an input device 1005, an output device 1006, and a bus 1007.

In the following description, the term “device” can be replaced with circuit, device, unit, or the like. The hardware configuration of the nudge management device 10 or 10A may be configured to include one or more devices illustrated in the drawing or may be configured to exclude some devices thereof.

The functions of the nudge management device 10 or 10A can be realized by reading predetermined software (program) to hardware such as the processor 1001 and the memory 1002 and causing the processor 1001 to execute arithmetic operations and to control communication using the communication device 1004 or to control at least one of reading and writing of data with respect to the memory 1002 and the storage 1003.

The processor 1001 controls a computer as a whole, for example, by causing an operating system to operate. The processor 1001 may be configured as a central processing unit (CPU) including an interface with peripherals, a controller, an arithmetic operation unit, and a register. For example, the control functions such as the acquisition unit 11 may be realized by the processor 1001.

The processor 1001 reads a program (a program code), a software module, data, or the like from at least one of the storage 1003 and the communication device 1004 to the memory 1002 and performs various processes in accordance therewith. As the program, a program that causes a computer to perform at least some of the operations described in the above-mentioned embodiment is used.

For example, the control functions such as the acquisition unit 11 may be realized by a control program which is stored in the memory 1002 and which operates in the processor 1001, and the other functional blocks may be realized in the same way. The various processes described above are described as being performed by a single processor 1001, but they may be simultaneously or sequentially performed by two or more processors 1001. The processor 1001 may be mounted as one or more chips. The program may be transmitted from a network via an electrical telecommunication line.

The memory 1002 is a computer-readable recording medium and may be constituted by, for example, at least one of a read only memory (ROM), an erasable programmable ROM (EPROM), an electrically erasable programmable ROM (EEPROM), and a random access memory (RAM). The memory 1002 may be referred to as a register, a cache, a main memory (a main storage device), or the like. The memory 1002 can store a program (a program code), a software module, and the like that can be executed to perform a radio communication method according to an embodiment of the present disclosure.

The storage 1003 is a computer-readable storage medium and may be constituted by, for example, at least one of an optical disc such as a compact disc ROM (CD-ROM), a hard disk drive, a flexible disk, a magneto-optical disc (for example, a compact disc, a digital versatile disc, or a Blu-ray (registered trademark) disc), a smart card, a flash memory (for example, a card, a stick, or a key drive), a floppy (registered trademark) disk, and a magnetic strip. The storage 1003 may be referred to as an auxiliary storage device. The storage media may be, for example, a database, a server, or another appropriate medium including the memory 1002 and/or the storage 1003.

The communication device 1004 is hardware (a transmitting and receiving device) that performs communication between computers via at least one of a wired network and a wireless network and is also referred to as, for example, a network device, a network controller, a network card, or a communication module.

The input device 1005 is an input device that receives an input from the outside (for example, a keyboard, a mouse, a microphone, a switch, a button, or a sensor). The output device 1006 is an output device that performs an output to the outside (for example, a display, a speaker, or an LED lamp). The input device 1005 and the output device 1006 may be configured as a unified body (for example, a touch panel).

The devices such as the processor 1001 and the memory 1002 are connected to each other via the bus 1007 for transmission of information. The bus 1007 may be constituted by a single bus or may be constituted by buses which are different depending on the devices.

The nudge management device 10 or 10A may be configured to include hardware such as a microprocessor, a digital signal processor (DSP), an application-specific integrated circuit (ASIC), a programmable logic device (PLD), or a field-programmable gate array (FPGA), and some or all of the functional blocks may be realized by the hardware. For example, the processor 1001 may be mounted using at least one piece of the hardware.

While the present disclosure has been described above in detail, it will be apparent to those skilled in the art that the present disclosure is not limited to the embodiments described in the present disclosure. The present disclosure can be altered and modified in various forms without departing from the gist and scope of the present disclosure defined by description in the appended claims. Accordingly, the description in the present disclosure is for exemplary explanation and does not have any restrictive meaning for the present disclosure.

The aspects/embodiments described in the present disclosure may be applied to a system using LTE (Long Term Evolution), LTE-A (LTE-Advanced), SUPER 3G, IMT-Advanced, 4G, 5G, FRA (Future Radio Access), NR (new Radio), W-CDMA (registered trademark), GSM (registered trademark), CDMA2000, UMB (Ultra Mobile Broadband), IEEE 802.11 (Wi-Fi (registered trademark)), IEEE 802.16 (WiMAX (registered trademark)), IEEE 802.20, UWB (Ultra-Wide Band), Bluetooth (registered trademark), or another appropriate system and/or a next-generation system which is extended based thereon.

The order of processing steps, the sequences, the flowcharts, and the like of the aspects/embodiments described above in the present disclosure may be changed unless conflictions arise. For example, in the methods described in the present disclosure, various steps are described as elements in the exemplary order, and the methods are not limited to the described specific order.

Information or the like which is input or output may be stored in a specific place (for example, a memory) or may be managed using a management table. Information or the like which is input or output may be overwritten, updated, or added. Information or the like which is output may be deleted. Information or the like which is input may be transmitted to another device.

Determination may be performed using a value (0 or 1) which is expressed in one bit, may be performed using a Boolean value (true or false), or may be performed by comparison between numerical values (for example, comparison with a predetermined value).

The aspects/embodiments described in the present disclosure may be used alone, may be used in combination, or may be switched during implementation thereof. Notifying of predetermined information (for example, notifying that “it is X”) is not limited to explicit notification, and may be performed by implicit notification (for example, notifying of the predetermined information is not performed).

Regardless of whether it is called software, firmware, middleware, microcode, hardware description language, or another name, software can be widely construed to refer to a command, a command set, a code, a code segment, a program code, a program, a sub program, a software module, an application, a software application, a software package, a routine, a sub routine, an object, an executable file, an execution thread, a sequence, a function, or the like.

Software, commands, and the like may be transmitted and received via a transmission medium. For example, when software is transmitted from a website, a server, or another remote source using at least one of wired technology such as a coaxial cable, an optical fiber cable, a twisted-pair wire, or a digital subscriber line (DSL.) and wireless technology such as infrared rays, radio waves, or microwaves, the wired technology and/or the wireless technology is included in definition of the transmission medium.

Information, signals, and the like described in the present disclosure may be expressed using one of various different techniques. For example, data, instructions, commands, information, signals, bits, symbols, and chips which can be mentioned in the overall description may be expressed by a voltage, a current, electromagnetic waves, a magnetic field or magnetic particles, a photo field or photons, or an arbitrary combination thereof.

Terms described in the present disclosure and terms required for understanding the present disclosure may be substituted with terms having the same or similar meanings.

Information, parameters, and the like described above in the present disclosure may be expressed using absolute values, may be expressed using values relative to predetermined values, or may be expressed using other corresponding information.

A communication terminal may also be referred to as a mobile communication terminal, a subscriber station, a mobile unit, a subscriber unit, a wireless unit, a remote unit, a mobile device, a wireless device, a wireless communication device, a remote device, a mobile subscriber station, an access terminal, a mobile terminal, a wireless terminal, a remote terminal, a handset, a user agent, a mobile client, a client, or several other appropriate terms by those skilled in the art.

The expression “based on” used in the present disclosure does not mean “based on only” unless otherwise described. In other words, the expression “based on” means both “based on only” and “based on at least.”

No reference to elements named with “first,” “second,” or the like used in the present disclosure generally limit amounts or order of the elements. These naming can be used in the present disclosure as a convenient method for distinguishing two or more elements. Accordingly, reference to first and second elements does not mean that only two elements are employed or that a first element precedes a second element in any form.

When the terms “include” and “including” and modifications thereof are used in the present disclosure, the terms are intended to have a comprehensive meaning similarly to the term “comprising.” The term “or” used in the present disclosure is not intended to mean an exclusive logical sum.

In the present disclosure, two or more of any devices may be included unless the context or technical constraints dictate that only one device is included.

In the entire present disclosure, singular terms include plural referents unless the context or technical constraints dictate that a unit is singular.

### REFERENCE SIGNS LIST

10, 10A Nudge management device (information processing device)
11 Acquisition unit
12 Storage unit
13 Abrasion deriving unit
14 Recovery deriving unit
15 Update unit
16 Abrasion feature generating unit
17 Behavioral modification estimating unit
18 Nudge selecting unit

1. An information processing device comprising:
a storage unit configured to store information of a nudge which is a mechanism for prompting a user to voluntarily adopt a desirable behavior;
an acquisition unit configured to acquire nudge intervention information which is information associated with intervention of a nudge; and
an abrasion deriving unit configured to derive nudge abrasion information indicating a degree of decrease in effect of the nudge associated with the nudge intervention information based on the nudge intervention information and to output the derived nudge abrasion information.

2. The information processing device according to claim 1, wherein the nudge intervention information includes at least information indicating whether the nudge has been viewed by the user in a predetermined period and information indicating whether the user has adopted a behavior promoted by the nudge.

3. The information processing device according to claim 1, wherein the acquisition unit additionally acquires user information which is associated with attributes of the user and which affects the decrease in effect of the nudge, and
wherein the abrasion deriving unit derives the nudge abrasion information in additional consideration of the user information.

4. The information processing device according to claim 1, wherein the acquisition unit additionally acquires environment information indicating an external environment state of the user in a predetermined period, and
wherein the abrasion deriving unit derives the nudge abrasion information in additional consideration of the environment information.

5. The information processing device according to claim 1, further comprising an update unit configured to update information of the nudge associated with the nudge intervention information and stored in the storage unit based on the nudge abrasion information.

6. The information processing device according to claim 5, wherein the storage unit stores information of a plurality of types of nudges,
wherein the abrasion deriving unit derives the nudge abrasion information for each of the plurality of types of nudges, and
wherein the update unit updates information of one of the nudges in consideration of both the nudge abrasion information of the one nudge included in the plurality of types of nudges and the nudge abrasion information of nudges similar to the one nudge.

7. The information processing device according to claim 5, further comprising a recovery deriving unit configured to derive nudge recovery information indicating a degree of recovery in effect of the nudge associated with the nudge intervention information based on the nudge intervention information,
wherein the update unit updates information of the nudge associated with the nudge intervention information in additional consideration of the nudge recovery information.

8. The information processing device according to claim 1, further comprising:
a behavioral modification estimating unit configured to estimate a behavioral modification probability which is a probability that the user adopts a behavior promoted by the nudge; and
a nudge selecting unit configured to select a nudge to be recommended to a user based on the behavioral modification probability,
wherein the storage unit stores information of the plurality of types of nudges,
wherein the abrasion deriving unit includes an abrasion feature generating unit configured to generate an abrasion feature quantity indicating the nudge abrasion information based on the nudge intervention information for each of the plurality of types of nudges,
wherein the behavioral modification estimating unit estimates the behavioral modification probability for each of the plurality of types of nudges based on the abrasion feature quantities of the plurality of types of nudges, and
wherein the nudge selecting unit selects a nudge with the highest behavioral modification probability as the nudge to be recommended to the user.

9. The information processing device according to claim 8, wherein the behavioral modification estimating unit generates an estimation model for estimating the behavioral modification probability for each of the plurality of types of nudges based on the abrasion feature quantities and user information which is information associated with attributes of a user, and
wherein the behavioral modification estimating unit estimates the behavioral modification probability for each of the plurality of types of nudges based on the estimation model.
